(19) United States
(12) Patent Application Publication
(10) Pub. No.: US 2024/0408097 A1
(43) Pub. Date: Dec. 12, 2024

(54) WEE1 INHIBITORS AND METHODS FOR TREATING CANCER

(71) Applicant: Recurium IP Holdings, LLC, San Diego, CA (US)

(72) Inventors: Ahmed Abdi Samatar, West Windsor, NJ (US); Fernando Donate, Santa Monica, CA (US); Petrus Rudolf de Jong, San Diego, CA (US); Laure Escoubet, San Diego, CA (US); Wen Liu, San Diego, CA (US); Philippe Pultar, Windham, NY (US); Dimitrios Voliotis, Madison, NJ (US); Kevin Duane Bunker, Escondido, CA (US); Peter Qinhua Huang, San Diego, CA (US); Jiali Li, San Diego, CA (US)

(21) Appl. No.: 18/389,654

(22) Filed: Dec. 19, 2023

Related U.S. Application Data
(63) Continuation of application No. PCT/US2022/034383, filed on Jun. 21, 2022.
(60) Provisional application No. 63/202,770, filed on Jun. 23, 2021.

(51) Int. Cl. A61K 31/519; A61P 35/00; C12Q 1/6886; G01N 33/574

(52) U.S. Cl. CPC A61K 31/519; A61P 35/00; C12Q 1/6886; G01N 33/57484; C12Q 2600/106; C12Q 2600/158; G01N 2333/4739

(57) ABSTRACT

Disclosed herein is a method of determining subject sensitivity to a WEE1 inhibitor, comprising obtaining or having obtained a biological sample from the subject, and performing or having performed at least one assay on the biological sample to determine if the subject has an altered function of CCNE1. Also disclosed are methods of treating cancer with a WEE1 inhibitor, comprising identifying a subject having (a) the cancer and (b) endogenous or altered function of CCNE1; and administering an effective amount of the WEE1 inhibitor, or a pharmaceutically acceptable salt thereof, to the subject.

### INCORPORATION BY REFERENCE TO PRIORITY APPLICATION

This application claims priority to U.S. Provisional Application Ser. No. 63/202,770, filed Jun. 23, 2021, which is hereby incorporated herein by reference in its entirety.

### BACKGROUND

### Field

The present application relates generally to compounds that are WEE1 inhibitors and methods of using them to treat conditions characterized by excessive cellular proliferation, such as cancer. It also relates to methods of identifying mutations in subjects with cancer and the subsequent treatment of those subjects with a WEE1 inhibitor.

### Description

DNA is constantly damaged from the environment. Light, chemicals, stress, and cellular replication lead to single- or double-stranded breakage along DNA's backbone. Typically, organisms defend against DNA damage by repair proteins that either re-connect, or re-synthesize damaged DNA. The correct functioning of these proteins are essential for life. The incorrect replacement of nucleotides into DNA can cause mutations (and other genetic alterations including but not limited to insertions, deletions, and frameshifts), genetic disease, and loss of protein function. The altogether loss of DNA repair can cause cell death, tumor progression, and cancer.

Cell cycle checkpoints are important for proper DNA repair, ensuring that cells do not progress with cellular replication until their genomic integrity is restored. WEE1 is a nuclear kinase involved in the G2-M cell-cycle checkpoint arrest for DNA repair before mitotic entry. Normal cells repair damaged DNA during G1 arrest. Cancer cells often have a deficient G1-S checkpoint and depend on a functional G2-M checkpoint for DNA repair. WEE1 is overexpressed in various cancer types. The compound known as ZN-c3 is a WEE1 inhibitor of the formula:

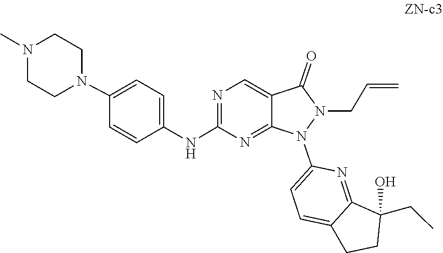 [SMILES: C=CCn1c(=O)c2cnc(Nc3ccc(N4CCN(C)CC4)cc3)nc2n1-c1ccc2c(n1)[C@@](O)(CC)CC2]

### SUMMARY

Various embodiments provide a method of determining subject sensitivity to a compound ZN-c3, or a pharmaceutically acceptable salt thereof, comprising:
obtaining or having obtained a biological sample from the subject; and
performing or having performed at least one assay on the biological sample to determine if the subject has an altered function of CCNE1.

Another embodiment provides a method of treating a cancer, comprising:
obtaining or having obtained a biological sample from a subject;
performing or having performed at least one assay on the biological sample to determine if the subject has altered function of CCNE1; and
administering an effective amount of a compound ZN-c3, or a pharmaceutically acceptable salt thereof, to the subject based upon results of the assay.

Another embodiment provides a method of treating a cancer, comprising:
identifying a subject having (a) the cancer and (b) endogenous or altered function of CCNE1; and
administering an effective amount of a compound ZN-c3, or a pharmaceutically acceptable salt thereof, to the subject.

Another embodiment provides a method of treating a cancer, comprising:
identifying a subject having (a) the cancer and (b) overexpression or altered function of CCNE1; and
administering an effective amount of a compound ZN-c3, or a pharmaceutically acceptable salt thereof, to the subject.

Another embodiment provides a method of treating cancer in a subject, comprising:
determining whether the subject is sensitized to treatment with a compound ZN-c3, or a pharmaceutically acceptable salt thereof, said determining comprising:
   obtaining or having obtained a biological sample from the subject; and
   performing or having performed at least one assay on the biological sample to determine if the subject has altered function of CCNE1; and
selecting a treatment protocol for the subject on the basis of the determination of whether the subject is sensitized to treatment with the compound ZN-c3 or pharmaceutically acceptable salt thereof.

These and other embodiments are described in greater detail below.

### BRIEF DESCRIPTION OF THE DRAWINGS

FIG. 1 illustrates Western blot results confirming CCNE1 overexpression in stable SKOV3 cell line compared to empty vector control SKOV3 cells (“Control”).

FIG. 2 is a plot showing that stable SKOV3 cells overexpressing CCNE1 are more sensitive to ZN-c3 compared to empty vector control SKOV3 cells (“Empty vector”) as measured by cell survival.

FIG. 3 shows a plot that depicts the percent cell density of OVCAR-3 ovarian cancer cells after treatment with a 10 point dose response of ZN-c3 for 72 hours. OVCAR-3 has amplification of CCNE1 (TABLE 2) and is sensitive to ZN-c3. Percent survival is calculated using cell titer glow analysis.

FIG. 4 shows a plot of the tumor volumes of 10 NOD/SCID mice inoculated subcutaneously on the right flank with 2×10⁷ OVCAR-3 cells and treated daily with vehicle or ZN-c3 (KP-2638) at 80 mg/kg. Tumors treated with ZN-c3 show significant tumor growth inhibition when compared against vehicle control.

FIG. 5 shows a plot of the tumor volumes of 10 BALB/c nude mice inoculated subcutaneously on the right flank with 1×10⁶ HCC1806 human triple negative breast cancer cells and treated daily with vehicle or ZN-c3 (KP-2638) at 80 mg/kg. HCC1806 cells have high copy number of CCNE1 (see TABLE 2). Tumors treated with ZN-c3 show significant tumor growth inhibition when compared against vehicle control.

### DETAILED DESCRIPTION

WEE1 is a tyrosine kinase that is a critical component of the ATR-mediated G2 cell cycle checkpoint control that prevents entry into mitosis in response to cellular DNA damage. ATR phosphorylates and activates CHK1, which in turn activates WEE1, leading to the selective phosphorylation of cyclin-dependent kinase 1 (CDK1) at Tyr15, thereby keeping the CDK1-cyclin B complex in an inactive state and halting cell-cycle progression. This process confers a survival advantage by allowing tumor cells time to repair damaged DNA prior to entering mitosis. Inhibition of WEE1 abrogates the G2 checkpoint, promoting cancer cells with DNA damage to enter into premature mitosis and undergo cell death via mitotic catastrophe. Therefore, WEE1 inhibition has the potential to sensitize tumors to DNA-damaging agents, such as cisplatin, and to induce tumor cell death. In addition, WEE1 activation can also lead to the selective phosphorylation of CDK2 at Tyr15, thereby regulating CDK2-cyclin A/E complexes which control the G1/S phase progression. Inhibition of WEE1 can result in excessive replication activity, thereby leading to replication catastrophe.

The compound ZN-c3 and pharmaceutically acceptable salts thereof are WEE1 inhibitors. The chemical structure of the compound ZN-c3 is depicted above. The compound ZN-c3 and pharmaceutically acceptable salts thereof can be prepared in various ways. See, e.g., WO 2019/173082.

Cyclin E1 (CCNE1) is involved in cell cycle regulation by binding to Cyclin-dependent kinases (CDKs), including CDK2, thereby promoting cell cycle progression.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, the term “about” has its usual meaning as understood by those skilled in the art and thus indicates that a value includes the inherent variation of error for the method being employed to determine a value, or the variation that exists among multiple determinations.

As used herein, the terms “modify” or “alter”, or any forms thereof, mean to modify, alter, replace, delete, substitute, remove, vary, or transform.

As used herein, the terms “function” and “functional” have their usual meaning as understood by those skilled in the art and thus refer to a biological, enzymatic, or therapeutic function.

As used herein, the term “endogenous” has its usual meaning as understood by those skilled in the art and thus refers to the native, or wild type property of a gene, protein, or cell. In some embodiments, the endogenous gene is the wild type sequence of said gene. In some embodiment, the endogenous protein is the wild type sequence of said protein. In some embodiments, the endogenous protein function is the wild type function and activity level of said protein. In some embodiments, the endogenous cell is the wild type cell.

The term “mutation” has its usual meaning as understood by those skilled in the art and refers to an alteration of genetic sequence. In some embodiments, cells have multiple mutations. In some embodiments, mutations are in coding regions of the genome. Mutations can range in size from a single nucleotide, to a large segment of the chromosome that includes multiple genes. In some embodiments, at least one mutation is silent, having no significant impact on gene expression or function. In some embodiments, at least one mutation has an impact on gene expression or function, such as gene amplification, overexpression, or enhanced copy number. In some embodiments, at least one mutation is silent, having no significant impact on protein expression or function. In some embodiments, at least one mutation has a small impact on protein expression or function. In some embodiments, at least one mutation has a moderate impact on protein expression or function. In some embodiments, at least one mutation has a large impact on protein expression or function. In some embodiments, at least one mutation prevents protein expression or function. Non-limiting examples of mutations include insertions, deletions, truncations, substitutions, duplications, translocations, and inversions. In some embodiments, mutations are “somatic,” or occurring in body cells and are not inheritable. In some embodiments, a subset of somatic cells in an organism have at least one mutation that other somatic cells do not have. In some embodiments, mutations are “germline,” or occurring in germ cells and are inheritable.

As disclosed herein, mutations can be monitored through a variety of sequencing, expression, or functional assays. Non-limiting examples include DNA sequencing, RNA sequencing, DNA hybridization, protein sequencing, targeted genomic sequencing, whole exome sequencing, whole genome sequencing, ATAC-sequencing, Sanger sequencing, PCR, qPCR, RT-PCR, RT-qPCR, Next Generation Sequencing, protein truncation test, DNA microarrays, heteroduplex analysis, denaturing gradient gel electrophoresis, nucleotide sequencing, single strand conformational polymorphism, restriction enzyme digestion assay, fluorescence in situ hybridization (FISH), comparative genomic hybridization, restriction fragment length polymorphism, amplification refractory mutation system PCR, nested PCR, multiplex ligation-dependent probe amplification, single strand conformational polymorphism, and oligonucleotide ligation assay. Mutations can also be monitored through a variety of antibody-based methods using biological samples including, but are not limited to, Western blotting, fluorescence activated cell sorting, immunofluorescence, immunohistochemistry, immunocytochemistry, immunoprecipitation, enzyme-linked immunosorbent assay, radioimmunoassays, and electrochemiluminescence assays.

The term “cancer” is used herein in its usual biological sense and understood by those skilled in the art. Thus, it can include the cancer of any cell type, such as but not limited to glioblastoma, astrocytoma, meningioma, craniopharyngioma, medulloblastoma, and other brain cancers, leukemia, skin cancer, adrenal cancer, anal cancer, bile duct cancer, bladder cancer, bone cancer, breast cancer, cervical cancer, colorectal cancer, endometrial cancer, esophagus cancer, eye cancer, gallbladder cancer, gastrointestinal cancer, Hodgkin lymphoma, hematological tumor, heme malignancy, Kaposi sarcoma, kidney cancer, laryngeal and hypopharyngeal cancer, liver cancer, lung cancer, lymphoma, mesothelioma, melanoma, multiple myeloma, neuroblastoma, nasopharyngeal cancer, ovarian cancer, osteosarcoma, pancreatic cancer, pituitary cancer, retinoblastoma, salivary gland cancer, stomach cancer, small intestine cancer, testicular cancer, thymus cancer, thyroid cancer, uterine cancer, uterine sarcoma, uterine serous carcinoma, vaginal cancer, vulvar cancer, Waldenstrom macroglobulinemia, Wilms tumor, solid tumor, or liquid tumor.

As used herein, the term “tumor” has its usual meaning as understood by those skilled in the art and refers to an abnormal growth of cells or tissue. In some embodiments, the tumor is benign. In some embodiments, the tumor is malignant. A tumor becomes a cancer when it metastasizes, or spreads to other areas of the body. The term “solid tumor” as used herein has its usual meaning as understood by those skilled in the art and refers to an abnormal mass of tissue that does not contain liquid areas or cysts. Non-limiting examples of solid tumors include sarcomas, carcinomas, or lymphomas. Many cancer tissues can form solid tumors, such as but not limited to breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas or ovarian cancer. The terms “cancer” and “tumor” may generally be used interchangeably unless the context clearly indicates that a more specific meaning is intended.

The term “cell” as used herein has its usual meaning as understood by those skilled in the art and can refer to any cell type. In some embodiments, said cells are mammalian cells. In some embodiments, said cells are human cells.

The terms “individual”, “subject”, or “patient” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea or pigs. In some embodiments, the subject can be human. In some embodiments, the subject can be a child and/or an infant. In other embodiments, the subject can be an adult.

The term “cancer treatment” as used herein has its usual meaning as understood by those skilled in the art and refers to a therapeutic modality (such as surgery and/or radiation) or an anti-cancer agent such as a small molecule, compound, protein, or other medicant that is used to treat, inhibit, or prevent cancer. Non-limiting examples of common classes of anti-cancer agents usable with any one or more of the alternatives described herein include alkylating agents, anti-EGFR antibodies, anti-Her-2 antibodies, antimetabolites, vinca alkaloids, platinum-based agents, anthracyclines, topoisomerase inhibitors, taxanes, antibiotics, immunomodulators, immune cell antibodies, interferons, interleukins, HSP90 inhibitors, anti-androgens, antiestrogens, anti-hypercalcaemia agents, apoptosis inducers, Aurora kinase inhibitors, Bruton's tyrosine kinase inhibitors, calcineurin inhibitors, CaM kinase II inhibitors, CD45 tyrosine phosphatase inhibitors, CDC25 phosphatase inhibitors, CHK kinase inhibitors, cyclooxygenase inhibitors, bRAF kinase inhibitors, cRAF kinase inhibitors, Ras inhibitors, cyclin dependent kinase inhibitors, cysteine protease inhibitors, DNA intercalators, DNA strand breakers, E3 ligase inhibitors, EGF Pathway Inhibitors, farnesyltransferase inhibitors, Flk-1 kinase inhibitors, glycogen synthase kinase-3 (GSK3) inhibitors, histone deacetylase (HDAC) inhibitors, I-kappa B-alpha kinase inhibitors, imidazotetrazinones, insulin tyrosine kinase inhibitors, c-Jun-N-terminal kinase (JNK) inhibitors, mitogen-activated protein kinase (MAPK) inhibitors, MDM2 inhibitors, MEK inhibitors, ERK inhibitors, MMP inhibitors, mTor inhibitors, NGFR tyrosine kinase inhibitors, p38 MAP kinase inhibitors, p56 tyrosine kinase inhibitors, PDGF pathway inhibitors, phosphatidylinositol 3-kinase inhibitors, phosphatase inhibitors, protein phosphatase inhibitors, PKC inhibitors, PKC delta kinase inhibitors, polyamine synthesis inhibitors, PTP1B inhibitors, protein tyrosine kinase inhibitors, SRC family tyrosine kinase inhibitors, Syk tyrosine kinase inhibitors, Janus (JAK-2 and/or JAK-3) tyrosine kinase inhibitors, retinoids, RNA polymerase II elongation inhibitors, serine/threonine kinase inhibitors, sterol biosynthesis inhibitors, VEGF pathway inhibitors, chemotherapeutic agents, alitretinon, altretamine, aminopterin, aminolevulinic acid, amsacrine, asparaginase, atrasentan, bexarotene, carboquone, demecolcine, efaproxiral, elsamitrucin, etoglucid, hydroxycarbamide, leucovorin, lonidamine, lucanthone, masoprocol, methyl aminolevulinate, mitoguazone, mitotane, oblimersen, omacetaxine, pegaspargase, porfimer sodium, prednimustine, sitimagene ceradenovec, talaporfin, temoporfin, trabectedin, or verteporfin.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. For WEE1 inhibitor compounds, those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the compounds described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Pharmaceutical Compositions

Some embodiments described herein relate to a pharmaceutical composition, that can include an effective amount of one or more compounds described herein (for example, a compound ZN-c3, or a pharmaceutically acceptable salt thereof) and a pharmaceutically acceptable carrier, diluent, excipient or combination thereof.

The term “pharmaceutical composition” refers to a mixture of one or more compounds and/or salts disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

The term “physiologically acceptable” refers to a carrier, diluent or excipient that does not abrogate the biological activity and properties of the compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a WEE1 inhibitor compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a WEE1 inhibitor compound, or a pharmaceutically acceptable salt thereof, can be administered orally.

One may also administer the WEE1 inhibitor compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

### Methods

Various embodiments provide a method of determining subject sensitivity to a compound ZN-c3, or a pharmaceutically acceptable salt thereof, comprising:
obtaining or having obtained a biological sample from the subject; and
performing or having performed at least one assay on the biological sample to determine if the subject has an altered function of CCNE1.

Another embodiment provides a method of treating a cancer, comprising:
obtaining or having obtained a biological sample from a subject;
performing or having performed at least one assay on the biological sample to determine if the subject has altered function of CCNE1; and
administering an effective amount of a compound ZN-c3, or a pharmaceutically acceptable salt thereof, to the subject based upon results of the assay.

Another embodiment provides a method of treating a cancer, comprising:
identifying a subject having (a) the cancer and (b) endogenous or altered function of CCNE1; and
administering an effective amount of a compound ZN-c3, or a pharmaceutically acceptable salt thereof, to the subject.

Another embodiment provides a method of treating a cancer, comprising:
identifying a subject having (a) the cancer and (b) overexpression or altered function of CCNE1; and
administering an effective amount of a compound ZN-c3, or a pharmaceutically acceptable salt thereof, to the subject.

Another embodiment provides a method of treating cancer in a subject, comprising:
determining whether the subject is sensitized to treatment with a compound ZN-c3, or a pharmaceutically acceptable salt thereof, said determining comprising:
   obtaining or having obtained a biological sample from the subject; and
   performing or having performed at least one assay on the biological sample to determine if the subject has altered function of CCNE1; and
selecting a treatment protocol for the subject on the basis of the determination of whether the subject is sensitized to treatment with the compound ZN-c3 or pharmaceutically acceptable salt thereof.

Various embodiments and implementation details of the above methods are described in greater detail elsewhere herein. All such embodiments and implementations will be understood to apply to all of the above methods unless the context clearly indicates otherwise.

In various embodiments of the above methods, the altered function of CCNE1 is CCNE1 gene amplification. In other embodiments, the altered function of CCNE1 is CCNE1 protein overexpression. In some embodiments, the altered function of CCNE1 results from CCNE1 gene mutation.

In various embodiments of the above methods, the cancer comprises one or more of the types of cancers (including tumors) described herein. In an embodiment, the cancer is a solid tumor. In an embodiment, the cancer is a heme malignancy. In some embodiments, the cancer is endometrial cancer, gallbladder cancer, or ovarian cancer. Examples of ovarian cancer include epithelial ovarian cancer, germ cell cancer, and stromal cancer. In an embodiment, the epithelial ovarian cancer is high grade serous ovarian cancer. In various embodiments of the above methods, the subject is a human.

A “functional assay” is a method to detect the activity of a gene, protein, or cell in response to a stimulus or insult. The specific functional assay performed depends on the specific mutation or mutations incorporated into the genome of the cell. For example, in various embodiments, a method as described herein may include performing or having performed at least one assay on a biological sample to determine if a subject has altered function of CCNE1 (or does not have altered function of CCNE1). Functional assays include, but are not limited to, kinase assays, transcription assays using, for example, reporter constructs, proliferation assays, apoptosis assays, migration/chemotaxis assays, nutrient sensitivity assay, agent (e.g., drug, chemotherapeutic agent, mutagen) or radiation sensitivity assays, nucleic acid-binding assay or protein-binding assay, all of which are within the ability of those of skill in the art.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The terms “administration” or “administering” as used herein have their usual meaning as understood by those skilled in the art and refer to providing or giving a subject an agent, such as a compound ZN-c3, or a pharmaceutically acceptable salt thereof, by any effective route. Exemplary routes of administration include, but are not limited to, oral, injection (such as intracranial, subcutaneous, intramuscular, intradermal, intraperitoneal, and intravenous), sublingual, rectal, transdermal, intranasal, vaginal, intraocular, or inhalation routes.

The terms “therapeutically effective amount” and “effective amount” are used to indicate an amount of an active compound, or pharmaceutical agent, that elicits the biological or medicinal response indicated. For example, a therapeutically effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The therapeutically effective amount of the ZN-c3 compounds and salts thereof disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound, or radiation, is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor. In the treatment of lung cancer (such as non-small cell lung cancer) a therapeutically effective amount is that amount that alleviates or eliminates cough, shortness of breath and/or pain. As another example, an effective amount, or a therapeutically effective amount of a WEE1 inhibitor is the amount which results in the reduction in WEE1 activity and/or phosphorylation (such as phosphorylation of CDC2, also known as CDK1). The reduction in WEE1 activity is known to those skilled in the art and can be determined by the analysis of WEE1 intrinsic kinase activity and downstream substrate phosphorylation.

The amount of the compound ZN-c3, or a pharmaceutically acceptable salt thereof, required for use in treatment will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

In general, however, a suitable dose will often be in the range of from about 0.05 mg/kg to about 10 mg/kg. For example, a suitable dose may be in the range from about 0.10 mg/kg to about 7.5 mg/kg of body weight per day, such as about 0.15 mg/kg to about 5.0 mg/kg of body weight of the recipient per day, about 0.2 mg/kg to 4.0 mg/kg of body weight of the recipient per day, or any amount in between. The compound may be administered in unit dosage form; for example, containing 1 to 500 mg, 10 to 100 mg, 5 to 50 mg or any amount in between, of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of the compound ZN-c3, or a pharmaceutically acceptable salt thereof, can be determined by comparing their in vitro activity, and in vivo activity in animal models. Such comparison can be done by comparison against an established drug, such as cisplatin and/or gemcitabine)

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

The compound ZN-c3, pharmaceutically acceptable salts thereof and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

### Example 1—Cell Culture Analysis Indicates that CCNE1 Overexpression Correlates with WEE1 Inhibitor Effectiveness in Halting Cellular Proliferation

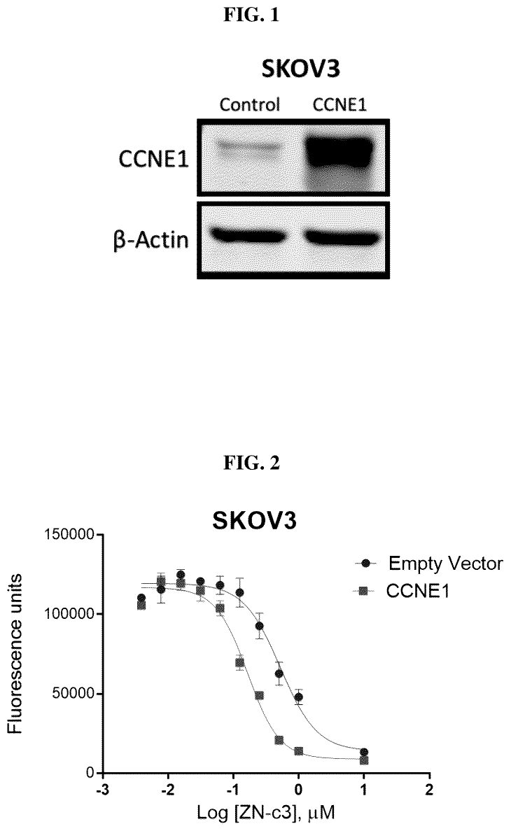

Cyclin E1 (CCNE1) is involved in cell cycle regulation by binding to Cyclin-dependent kinases (CDKs), including CDK2, thereby promoting cell cycle progression. The ovarian cancer cell culture line SKOV3, which has low endogenous expression levels of CCNE1, was transduced with lentiviral vector with CCNE1 or empty vector control. Stable SKOV3 cell lines were established by puromycin selection. CCNE1 overexpression in the stable SKOV3 cell line compared to empty vector control cells was confirmed by Western blotting (FIG. 1). These cells were then treated with increasing concentrations of ZN-c3 over 6 days and monitored for cell survival (FIG. 2). Compared to control cells, cells overexpressing CCNE1 function were 3.1-fold more sensitive to WEE1 inhibition (TABLE 1). These results indicate that CCNE1 amplifications enhance the effectiveness of WEE1 inhibitors in cancer cells.

TABLE 1

| | IC50 (μM) |
|---|---|
| Ctrl | 0.5217 |
| CCNE1 | 0.1655 |

TABLE 1 summarizes the statistical analysis (IC₅₀, μM) of the results of the cell culture experiment depicted in FIG. 2.

### Example 2—Tumor Cell Growth Analysis Indicates that Cell Lines which have High Copy Number of CCNE1 Correlates with WEE1 Inhibitor Effectiveness in Halting Cellular Proliferation

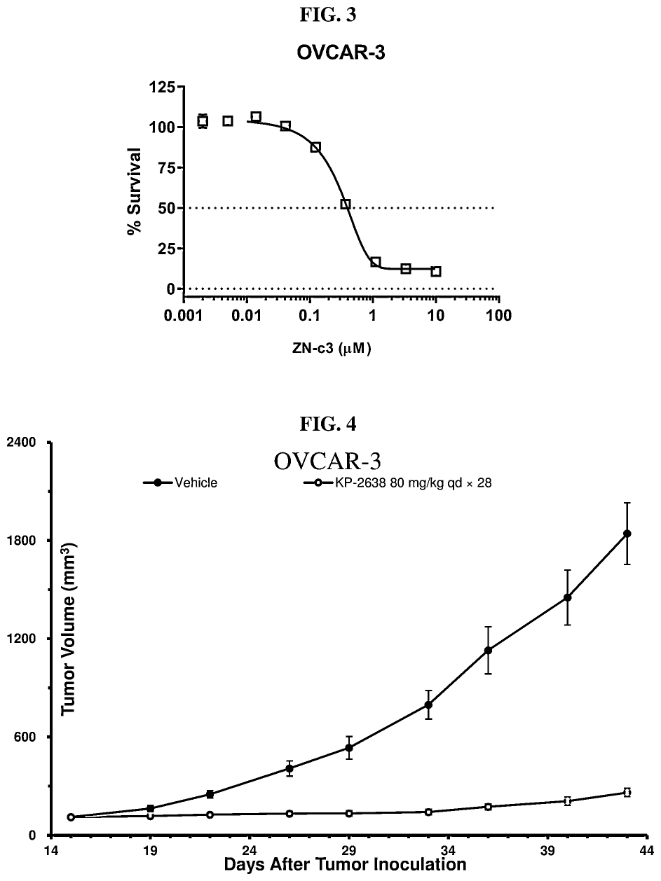

The OVCAR-3 human ovarian cancer cell line has amplified copy number of CCNE1 (TABLE 2) which correlates with high expression of CCNE1 protein. OVCAR-3 cells were treated with increasing concentrations of ZN-c3 over 72 hours and monitored for cell survival (FIG. 3). ZN-c3 treated cells demonstrated an IC50 sensitivity of 395 nM (TABLE 3). These results indicate that high copy number of CCNE1 leads to sensitivity to ZN-c3 treatment.

TABLE 2

| Cell Line | CCNE1 Copy Number |
|---|---|
| OVCAR-3 | 11 |
| HCC1806 | 6 |

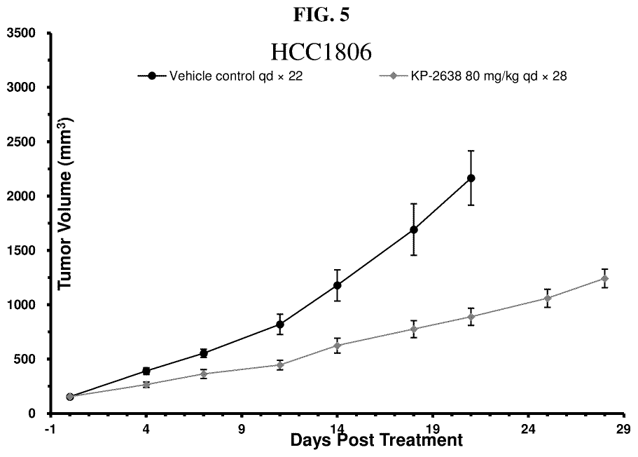

TABLE 2 summarizes the copy number of CCNE1 in cell lines used in FIG. 3, FIG. 4, and FIG. 5. Data was accessed from the Catalogue Of Somatic Mutations In Cancer (COSMIC) database.

TABLE 3

| Compound | IC₅₀ (μM) |
|---|---|
| ZN-c3 | 0.395 |

TABLE 3 summarizes the statistical analysis (IC₅₀, μM) of the results of the cell culture experiment depicted in FIG. 3.

### Example 3—In Vivo Tumor Growth Inhibition Analysis Indicates that Cell Lines which have High Copy Number of CCNE1 Correlates with WEE1 Inhibitor Effectiveness in Halting Tumor Growth

20 million OVCAR-3 cells were inoculated into the right flank of ten 6-8 week old female NOD/SCID mice. Animals were randomized and treatment started when the mean tumor volume reached 111 mm³. Animals were treated with vehicle (20% HP-3-CD) or 80 mg/kg ZN-c3 daily and tumor volumes and body weight were measured twice weekly. OVCAR-3 has amplified copy number of CCNE1 (TABLE 2). Compared against mice treated with vehicle, ZN-c3 treated mice show significantly reduced tumor growth after 28 days (FIG. 4). These results indicate that high copy number of CCNE1 leads to sensitivity to ZN-c3 treatment.

1×10⁶ HCC1806 cells were inoculated into the right flank of ten 6-8 week old female BALB/c nude mice. Animals were randomized and treatment started when the mean tumor volume reached 155 mm³. Animals were treated with vehicle (20% HP-3-CD) or 80 mg/kg ZN-c3 daily and tumor volumes and body weight were measured twice weekly. The HCC1806 human triple negative breast cancer cell line has amplified copy number of CCNE1 (TABLE 2). Compared against mice treated with vehicle, ZN-c3 treated mice show significantly reduced tumor growth after 28 days (FIG. 5). These results indicate that high copy number of CCNE1 leads to sensitivity to ZN-c3 treatment.

### Example 4—Clinical Trials Demonstrate Superior Ability of WEE1 Inhibitors to Treat Cancer in Subjects with Cyclin E1 (CCNE1) Gene Amplification

The effectiveness of WEE1 inhibitors in human subjects having CCNE1 gene amplification was assessed through clinical trials. A human subject (Patient 1) with Stage IVB Gallbladder cancer who had received 4 prior therapies was allowed to participate on clinical trial ZN-c3-005 based on a pre-existing genomic report showing an amplification in the CCNE1 gene.

Patient 1 began treatment with 300 mg QD of ZN-c3. Patient 1 was assessed for response to ZN-c3 approximately 6 weeks later. Tumor progression was monitored using tumor imaging analysis which showed a 38% reduction in measurable tumors. These results illustrate the enhanced effectiveness of WEE1 inhibition in a human subject having CCNE1 gene amplification under a clinical setting.

1. (canceled)

2. A method of treating a cancer, comprising:
obtaining or having obtained a biological sample from a subject;
performing or having performed at least one assay on the biological sample to determine if the subject has altered function of CCNE1; and
administering an effective amount of a compound having the structure 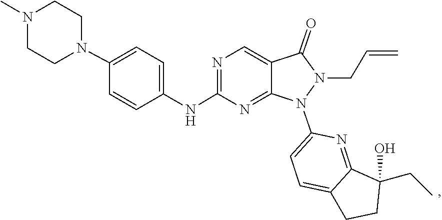 [SMILES: C=CCn1c(=O)c2cnc(Nc3ccc(N4CCN(C)CC4)cc3)nc2n1-c1ccc2c(n1)[C@@](O)(CC)CC2]
or a pharmaceutically acceptable salt thereof, to the subject based upon results of the assay.

3. (canceled)

4. A method of treating a cancer, comprising:
identifying a subject having (a) the cancer and (b) overexpression or altered function of CCNE1; and
administering an effective amount of a compound having the structure 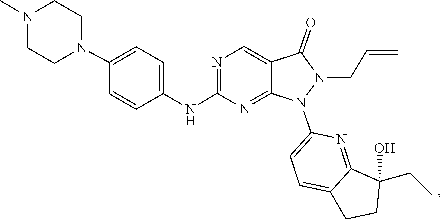 [SMILES: C=CCn1c(=O)c2cnc(Nc3ccc(N4CCN(C)CC4)cc3)nc2n1-c1ccc2c(n1)[C@@](O)(CC)CC2]
or a pharmaceutically acceptable salt thereof, to the subject.

5. A method of treating cancer in a subject, comprising:
determining whether the subject is sensitized to treatment with a compound having the structure 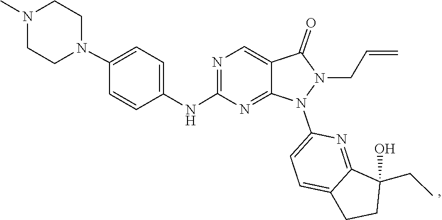 [SMILES: C=CCn1c(=O)c2cnc(Nc3ccc(N4CCN(C)CC4)cc3)nc2n1-c1ccc2c(n1)[C@@](O)(CC)CC2]
or a pharmaceutically acceptable salt thereof, said determining comprising:
obtaining or having obtained a biological sample from the subject; and
performing or having performed at least one assay on the biological sample to determine if the subject has altered function of CCNE1; and
selecting a treatment protocol for the subject on the basis of the determination of whether the subject is sensitized to treatment with the compound having the structure 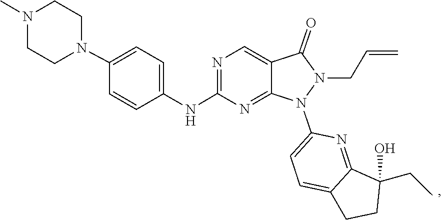 [SMILES: C=CCn1c(=O)c2cnc(Nc3ccc(N4CCN(C)CC4)cc3)nc2n1-c1ccc2c(n1)[C@@](O)(CC)CC2]
or pharmaceutically acceptable salt thereof.

6. The method of claim 2, wherein the altered function of CCNE1 is CCNE1gene amplification.

7. The method of claim 2, wherein the altered function of CCNE1 is CCNE1protein overexpression.

8. (canceled)

9. The method of claim 2, wherein the cancer is selected from the group consisting of a solid tumor, a heme malignancy, endometrial cancer, gallbladder cancer, and ovarian cancer.

10. (canceled)

11. The method of claim 9, wherein the ovarian cancer is epithelial ovarian cancer, germ cell cancer, or stromal cancer.

12. The method of claim 11, wherein the epithelial ovarian cancer is high grade serous ovarian cancer.

13. (canceled)

14. The method of claim 4, wherein the altered function of CCNE1 is CCNE1 gene amplification.

15. The method of claim 4, wherein the altered function of CCNE1 is CCNE1 protein overexpression.

16. The method of claim 5, wherein the altered function of CCNE1 is CCNE1 gene amplification.

17. The method of claim 5, wherein the altered function of CCNE1 is CCNE1 protein overexpression.

18. The method of claim 4, wherein the cancer is selected from the group consisting of a solid tumor, a heme malignancy, endometrial cancer, gallbladder cancer, and ovarian cancer.

19. The method of claim 18, wherein the ovarian cancer is epithelial ovarian cancer, germ cell cancer, or stromal cancer.

20. The method of claim 5, wherein the cancer is selected from the group consisting of a solid tumor, a heme malignancy, endometrial cancer, gallbladder cancer, and ovarian cancer.

21. The method of claim 20, wherein the ovarian cancer is epithelial ovarian cancer, germ cell cancer, or stromal cancer.

22. The method of claim 6, wherein the CCNE1 copy number is at least 6.

23. The method of claim 14, wherein the CCNE1 copy number is at least 6.

24. The method of claim 16, wherein the CCNE1 copy number is at least 6.
